(19) United States
(12) Patent Application Publication
(10) Pub. No.: US 2019/0046109 A1
(43) Pub. Date: Feb. 14, 2019

(54) Motorized Window Shade System For Use As An Alarm Clock Or To Control Sleep Cycles

(71) Applicant: Mike Lewis, San Francisco, CA (US)

(72) Inventor: Mike Lewis, San Francisco, CA (US)

(21) Appl. No.: 15/675,485

(22) Filed: Aug. 11, 2017

(51) Int. Cl. A61B 5/00; A61M 21/02; E06B 9/68; E06B 9/32

(52) U.S. Cl. CPC A61B 5/4812; A61M 21/02; A61M 2021/0044; E06B 9/32; E06B 2009/6809; E06B 9/68

(57) ABSTRACT

The herein disclosed motorized window shade system and associated methods for controlling a window shade provide a user with the ability to reduce ambient light during predetermined sleep times and provide the user with the ability to wake up to natural sunlight on a schedule that suits the user's needs. The present invention may be utilized in (and for) all possible sleep situations, but may be utilized most advantageously in residential bedrooms, child nurseries, and hotel rooms to provide users with fully adjustable and customizable sleep cycle(s) delineated by natural light as controlled by the herein disclosed motorized window shade system.

An exemplary embodiment of the herein disclosed motorized window shade system comprises: a shade housing surrounding an extendable window shade, wherein the shade housing includes a processor; a motor module for controllably adjusting a shade height of the extendable window shade; a wireless communication device; and a power supply.

An exemplary embodiment of the herein disclosed method for controlling a window shade so as to control a user's sleep cycles may comprise: connecting to the user's mobile device; communicating a current setting from an onboard database to the user's mobile device; receiving a desired setting from the user's mobile device; updating the current setting from the onboard database to an updated current setting on the onboard database based upon the desired setting; utilizing the updated current setting on the onboard database to control the window shade so as to control the user's sleep cycles.

### BRIEF DESCRIPTION OF THE INVENTION

The present invention relates to motorized window shades, and more particularly to a motorized window shade system for use as an alarm clock or to control sleep cycles. The present invention may also be described as methods for controlling a window shade so as to control a user's sleep cycles and/or to wake up a user at a predetermined time or in a predetermined way.

The herein disclosed motorized window shade system and associated methods for controlling a window shade provide a user with the ability to reduce ambient light during predetermined sleep times and provide the user with the ability to wake up to natural sunlight on a schedule that suits the user's needs. The present invention may be utilized in (and for) all possible sleep situations, but may be utilized most advantageously in residential bedrooms, child nurseries, and hotel rooms to provide users with fully adjustable and customizable sleep cycle(s) delineated by natural light as controlled by the herein disclosed motorized window shade system.

An exemplary embodiment of the herein disclosed motorized window shade system comprises: a shade housing surrounding an extendable window shade, wherein the shade housing includes a processor; a motor module for controllably adjusting a shade height of the extendable window shade; a wireless communication device; and a power supply. Alternative embodiments of the herein disclosed motorized window shade system may additionally comprise a speaker for emitting one or more sounds.

An exemplary embodiment of the herein disclosed method for controlling a window shade so as to control a user's sleep cycles may comprise: connecting to the user's mobile device; communicating a current setting from an onboard database to the user's mobile device; receiving a desired setting from the user's mobile device; updating the current setting from the onboard database to an updated current setting on the onboard database based upon the desired setting; utilizing the updated current setting on the onboard database to control the window shade so as to control the user's sleep cycles. An alternative embodiment of the herein disclosed method for controlling a window shade so as to control a user's sleep cycle may comprise: connecting to a cloud server; communicating a current setting from an onboard database to the cloud server; the cloud server receiving a desired setting from the user's mobile device; storing the desired setting in the cloud server; receiving the desired setting from the cloud server; updating the current setting from the onboard database to an updated current setting on the onboard database based upon the desired setting; utilizing the updated current setting on the onboard database to control the window shade so as to control the user's sleep cycles.

### CROSS-REFERENCES TO RELATED APPLICATIONS

Not applicable.

### STATEMENTS AS TO THE RIGHTS TO INVENTIONS MADE UNDER FEDERALLY SPONSORED RESEARCH OR DEVELOPMENT

Not applicable.

### REFERENCE TO A “SEQUENCE LISTING,” A TABLE, OR A COMPUTER PROGRAM LISTING APPENDIX SUBMITTED ON A COMPACT DISK

Not applicable.

### BACKGROUND OF THE INVENTION

Significant research has uncovered evidence that human sleep is very sensitive to light. Ideally, ambient light should be reduced during sleeping time. And ideally, natural sunlight should be used to wake up and thereby end the sleep cycle.

Motorized window shade system can help adjust window shades, and thereby adjust the amount of light allowed through the window at any particular time. Currently available motorized shade system fall into two categories. “Dumb” motorized shade systems, which require a manual user action to activate the motor, which then controls the shades to move up or down depending on the user's active instructions to the motor. And “Smart” motorized shade systems, which generally are part of a larger whole-home automation system. So-called “smart” motorized shade systems are capable of opening and closing automatically, but the currently available systems are not dedicated to precisely controlling the window shade system. Because these “smart” systems are designed for the whole-home, the ability to precisely control times, speeds, and shade heights are non-existent, and therefore it is difficult to utilize such a whole-home system as an alarm-clock to regulate sleep cycles using natural light entering through a shade-controlled window.

There is a device on the market that purports to use a motorized device to precisely control a window shade. The Axis device (www.helloaxis.com) is a small device that may be placed onto the beaded chain or cord loop that manually controls the height of the shades. The Axis device then uses its motor to turn a gear which in turn pulls on the beaded chain or cord loop, to adjust the height of the shades. But the Axis device is an add-on for use with a user's current window shades. The Axis device does not include an extendable window shade as part of the integrated system.

There is a need, therefore, to provide an individually configurable motorized window shade system that may be precisely configured to control the user's sleep cycle.

### BRIEF DESCRIPTION OF THE SEVERAL VIEWS OF THE DRAWING

FIG. 1 illustrates a general overview of an exemplary embodiment of a motorized window shade system, in accordance with the present invention;

FIG. 2 illustrates a general overview of an alternative embodiment of a motorized window shade system, wherein the motorized window shade system includes a processor power supply for supplying power to the processor and to the wireless communication device, and a shade power supply for supplying power to the motor module, in accordance with the present invention;

FIG. 3 is a schematic flowchart of a method for controlling a window shade so as to control a user's sleep cycle, in accordance with the present invention; and

FIG. 4 is a schematic flowchart of a method for controlling a window shade so as to control a user's sleep cycle, in accordance with an alternative embodiment of the present invention wherein a cloud server is utilized.

### DETAILED DESCRIPTION OF THE INVENTION

The herein disclosed motorized window shade system and associated methods for controlling a window shade provide a user with the ability to reduce ambient light during predetermined sleep times and provide the user with the ability to wake up to natural sunlight on a schedule that suits the user's needs. The present invention may be utilized in (and for) all possible sleep situations, but may be utilized most advantageously in residential bedrooms, child nurseries, and hotel rooms to provide users with fully adjustable and customizable sleep cycle(s) delineated by natural light as controlled by the herein disclosed motorized window shade system. An exemplary embodiment of the herein disclosed motorized window shade system comprises: a shade housing surrounding an extendable window shade, wherein the shade housing includes a processor; a motor module for controllably adjusting a shade height of the extendable window shade; a wireless communication device; and a power supply. Alternative embodiments of the herein disclosed motorized window shade system may additionally comprise a speaker for emitting one or more sounds.

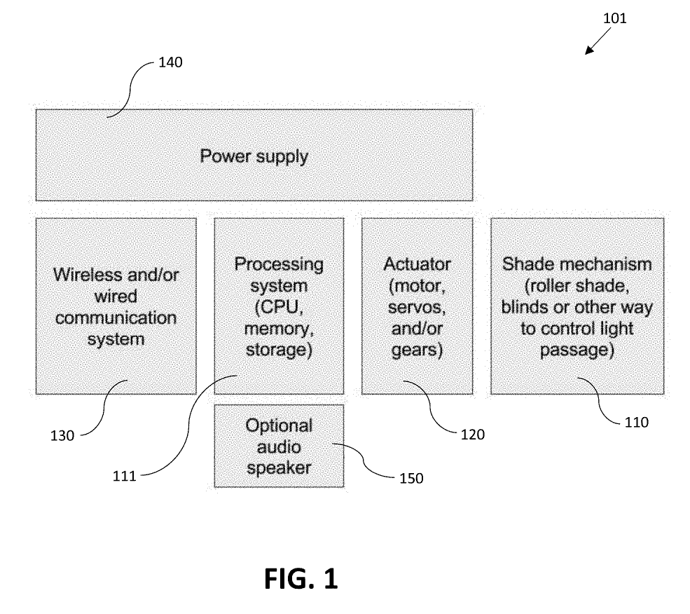

Referring to FIG. 1, a general overview of an exemplary embodiment of a motorized window shade system 101 is shown. The herein disclosed motorized window shade system 101 includes shade housing 110 surrounding an extendable window shade, processor 111 (which may be integrated into shade housing 110), motor module 120, wireless communication device 130, and power supply 140. An alternative embodiment of the present invention may include audio speaker 150 for emitting one or more sounds.

Shade housing 110 may be a cylindrical tube, a rectangular-shaped box-like enclosure, or any other type of enclosure known in the art that is capable of supporting an extendable window shade or a portion of an extendable window shade, such as an anchor for the extendable window shade. For example, shade housing 110 may be a cylindrical tube having an axis anchored within for holding (or supporting) a rolling-type shade that may spin on the axis and thereby extend and/or retract the shade across the span of a window. Shade housing 110 may alternatively functionally and extendably support other types of shades, such as vertical blinds or venetian blinds, for example. Furthermore, the phrase extendable window shade is intended to include all alternative types of shades capable of blocking light, including vertical blinds or venetian blinds for example. And while the shade housing is described as surrounding an extendable window shade, shade housing 110 may form an aperture for the extendable window shade to extend through in order to traverse the window.

Shade housing 110 may include processor 111. Processor 111 may be any type of processor known in the art, and may include a CPU and a storage (such as a hard drive, for example) capable of storing one or more settings, or sets of settings, in an onboard database. For example, the onboard database associated with processor 111 (or with the storage of processor 111) may be capable of storing a current setting, a desired setting, and an updated current setting. Processor 111 may control and/or operate various components of the herein disclosed motorized window shade system, such as the extendable window shade, motor module 120, wireless communication device 130, power supply 140, and the speaker 150.

The herein disclosed motorized window shade system 101 includes motor module 120 for controllably adjusting a shade height of the extendable window shade. Motor module 120 may include one or more motors, one or more servos, and/or one or more gears. Motor module 120 may be controlled by processor 111 to adjust a height of the extendable window shade so as to control the amount of light coming through a user's window. The shade can be raised, or lowered, to a desired shade height. Movement of the extendable shade may be in increments (stopping at various predetermined shade heights) as controlled by processor 111, or may be continuous (traversing the entire window) as controlled by processor 111. For example, motor module 120 may move the extendable window shade slowly over the entire window, so that a user's sleep cycle may be controlled in that the window will be opened slowly over a predetermined (or selected) time period.

Wireless communication device 130 may be any type of wireless communication known in the art capable of communicating with nearby (or internet-connected) wireless devices. For example, wireless communication device 130 may include one or more of the following known wireless technologies: WiFi, Bluetooth, Radio Frequency (RF), or any other type of wireless technology. Wireless communication device 130 may be utilized by the herein disclosed system to communicate (or transmit) one or more settings from a user's mobile device to processor 111. Alternatively, wireless communication device 130 may communicate directly with a cloud server having a cloud database for storing one or more settings. In an alternative embodiment of the present invention, a hard wire data transfer system may be utilized to communicate with a cloud server and/or a remotely-located device over a hard line.

The herein disclosed motorized window shade system 101 includes power supply 140. Power supply 140 may be a battery or a series of batteries, as is known in the art, connectively coupled to motorized window shade system 101. Alternatively, power supply 140 may include an AC wall adaptor for powering motorized window shade system 101 via a standard wall electrical outlet. Those skilled in the art will recognize that many types of power supplies may be appropriate for powering the herein disclosed system, and all such power supplies are intended to be included herein. For an alternative embodiment of motorized window shade system 101, power supply 140 may include a solar panel for producing electrical power from light. In this alternative embodiment, the electrical power produced by the solar panel may be stored in a rechargeable battery. Such a solar panel may be a mini solar panel, and may be positioned against the window so as to receive light through the window even at times when window shade system 101 has positioned the extendable window shade to a shade height of fully-closed. In other words, power supply 140 may include a mini solar panel positioned between the window and the extendable window shade so that the light-receiving solar panels are outward-facing, thus enabling reception of light by the solar panel regardless of the position of the extendable window shade.

An alternative embodiment of herein disclosed motorized window shade system may include audio speaker 150 for emitting one or more sounds. Audio speaker 150 may be any speaker capable of emitting one or more sounds. In this alternative embodiment, emitting of one or more sounds may be combined with controllably adjusting the shade height of the extendable window shade to more thoroughly (or more precisely) control the user's sleep cycle. For example, a user may set the herein disclosed motorized window system so that at a predetermined time the motor module will begin adjusting the shade height (so as to begin opening the window shade to allow light to enter the window) and the speaker will begin to emit a wake-up alarm sound. Speaker 150 may be controlled by processor 111. The storage of processor 111 may additionally store one or more digital sounds that may be emitted by speaker 150.

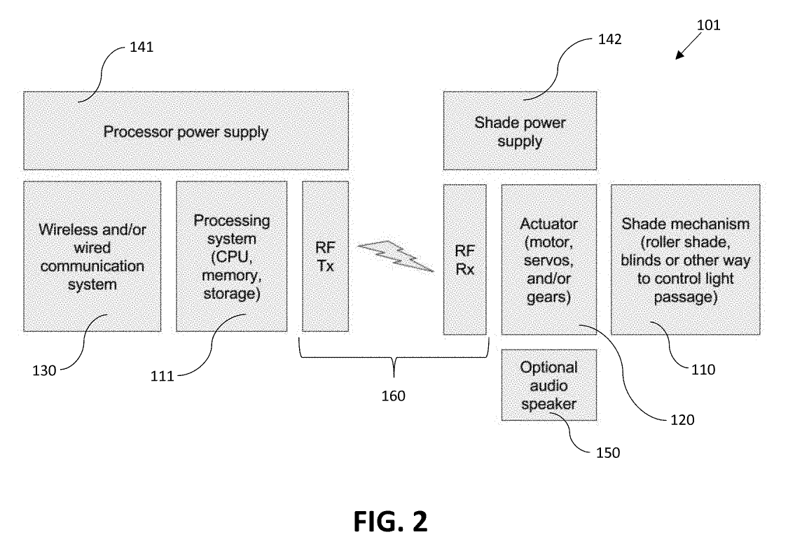

Referring to FIG. 2, a general overview of an alternative embodiment of motorized window shade system 101, wherein the motorized window shade system includes a processor power supply for supplying power to the processor and to the wireless communication device, and a shade power supply for supplying power to the motor module, is shown. The alternative embodiment of FIG. 2 includes shade housing 110 for surrounding an extendable window shade and having processor 111, motor module 120 for controllably adjusting a shade height of the extendable window shade, and wireless communication device 130 for communicating with a user's mobile device or a cloud server. In this alternative embodiment, both the processor and the motor module are independently powered. As seen in FIG. 2, processor 111 and wireless communication device 130 may be powered by processor power supply 141, while motor module 120 may be powered by shade power supply 142. Both processor power supply 141 and shade power supply 142 may be any type of power supply known in the art. In this alternative embodiment, the physically separate processor power supply 141 (and connectively coupled wireless communication device 130 and processor 111) may be wirelessly linked to shade power supply 142 (and the connectively coupled motor module 120). This wireless link may be a radio frequency (RF) link, as is known in the art.

Alternative embodiments of the herein disclosed motorized window shade system 101 may include a photosensor, positioned on the window, for the purpose of monitoring brightness levels (or light levels) of the environment outside the window. Such a photosensor may monitor an outside light level and utilize the light level to adjust one or more setting of motorized window shade system 101. For example, if it is a particularly cloudy day outside the photosensor may read a lower than average light level. Processor 111 may utilize this light level to adjust one or more setting by, for example, determining that it should control the extendable window shade to traverse the window to a full-open position because of the low light levels, as opposed to traversing the window to a half-open position as would normally be done under average light levels as monitored by the photosensor. In another example, if the photosensor reads a light level indicating that the outside environment includes exceptionally bright light, processor 111 may utilize this above-average light level to control the extendable window shade to traverse the window to a quarter-open position (a less open position than usual) to account for the increased light levels as monitored by the photosensor. Processor 111 may compare the light level received from the photosensor to historical data on light levels (which may be stored in the onboard database or may be accessible to the processor through communication with a remote database, which may be in communication with the processor via the wireless communication device), so as to intelligently adjust one or more settings based upon any divergence between the current light level as monitored by the photosensor and the historical light level data. Any type of photosensor capable of monitoring a light level and transmitting the light level to a processor may be utilized, as will be appreciated by those skilled in the art.

Methods for controlling a window shade so as to control a user's sleep cycle are also herein disclosed. An exemplary embodiment of the herein disclosed method for controlling a window shade so as to control a user's sleep cycles may comprise: connecting to the user's mobile device; communicating a current setting from an onboard database to the user's mobile device; receiving a desired setting from the user's mobile device; updating the current setting from the onboard database to an updated current setting on the onboard database based upon the desired setting; utilizing the updated current setting on the onboard database to control the window shade so as to control the user's sleep cycles.

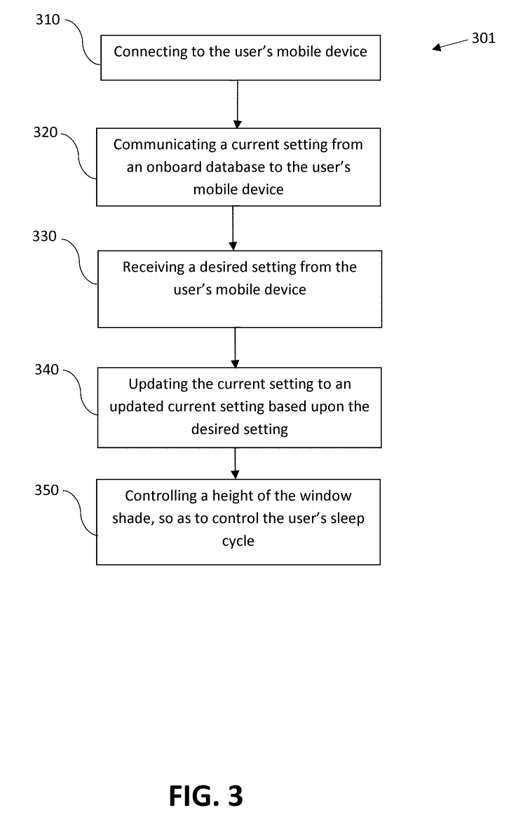

Referring to FIG. 3, a schematic flowchart of a method for controlling a window shade so as to control a user's sleep cycle is shown. The herein disclosed method 301 for controlling a window shade to control a user's sleep cycle includes step 310 connecting to the user's mobile device. System 101 may utilize communication device 130 to connect to a user's mobile device, as is known in the art. Step 320 then includes communicating a current setting from an onboard database to the user's mobile device. A current setting may include settings for how the user's sleep cycle is to be controlled, and may include, for example, one or more times, one or more shade heights, one or more speeds for traversing the window to the shade height, and/or one or more sounds for emitting by speaker 150.

Method 301 for controlling a window shade further includes step 330 receiving a desired setting from the user's mobile device. The user may wish to adjust, edit, or change the setting for how the user's sleep cycle is to be controlled. The user may input his or her desired setting either into his or her mobile device, or directly at processor 111 of the herein disclosed motorized window shade system 101. Step 330 involves receiving this desired setting from the user's mobile device via wireless communication device 130.

Method 301 for controlling a window shade further includes step 340 updating the current setting from the onboard database to an updated current setting on the onboard database based upon the desired setting. Step 340 involves changing the current setting to an updated current setting, in order to reflect the desired setting input by the user and received by system 101 during step 330.

Finally, method 301 for controlling a window shade includes step 350, controlling a height of the window shade by utilizing the updated current setting on the onboard database, so as to control the user's sleep cycle. A height of the window shade may also be referred to as a shade height. Step 350 involves processor 111 controlling motor module 120 so that the extendable window shade is adjusted according to the updated current setting. For example, if the updated current setting specifies that the extendable window shade is to be raised to a shade height of half-open at a time of 6:30 AM at full speed, then processor 111 will control motor module 120 to raise the extendable window shade to a shade height of half-open at full speed at 6:30 AM.

An alternative embodiment of the herein disclosed method for controlling a window shade so as to control a user's sleep cycle may comprise connecting to a cloud server; communicating a current setting from an onboard database to the cloud server; the cloud server receiving a desired setting from the user's mobile device; storing the desired setting in the cloud server; receiving the desired setting from the cloud server; updating the current setting from the onboard database to an updated current setting on the onboard database based upon the desired setting; utilizing the updated current setting on the onboard database to control the window shade so as to control the user's sleep cycles. Utilizing a cloud server as an intermediary between the user's mobile device and the herein disclosed system 101 provides the benefit of allowing system 101 to operate even if the user's mobile device is unable to connect directly to communication device 130 of system 101, or if the user's mobile device is unable to connect with the cloud server at the same time as system 101, because the one or more settings may be stored in the cloud server.

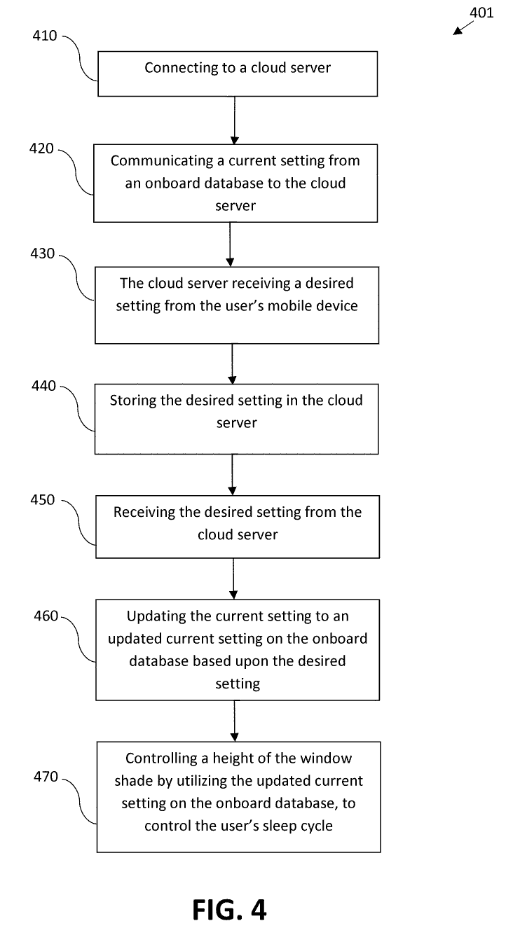

Referring to FIG. 4, alternative method 401 for controlling a window shade so as to control a user's sleep cycle utilizing a cloud server is shown. The purpose of including a cloud server into alternative method 401 is so that the user's mobile device and the herein disclosed motorized window shade system 101 need not be directly connected. The cloud server can be utilized to store and pass one or more settings between the user's mobile device and motorized window shade system 101, even when the user's mobile device and the herein disclosed system 101 are not within proximity of each other. This communication, via the cloud server intermediary, may be carried out asynchronously so that a situation wherein motorized window shade system 101 is temporarily unable to connect to the internet cloud server does not render the system inoperable.

The herein disclosed method 401 for controlling a window shade to control a user's sleep cycle includes step 410 connecting to a cloud server. The cloud server may be any internet-connected cloud server having a cloud database for storing one or more settings. Then, step 420 includes communicating a current setting from an onboard database to the cloud server. In this way, the cloud server includes the most current setting of system 101. The communication of step 420 may utilize wireless communication device 130. Step 430 then involves the cloud server receiving a desired setting from the user's mobile device. The user's mobile device may utilize a downloaded application (or alternatively an internet website) to receive input of the user's desired setting. This desired setting is then received by the cloud server and stored on the cloud server in a cloud database, in step 440.

The herein disclosed method 401 for controlling a window shade to control a user's sleep cycle further includes step 450 receiving the desired setting from the cloud server. In step 450, herein disclosed system 101 utilizes communication device 130 to receive the desired setting from the cloud server. Once received in step 450, step 460 then involves updating the current setting (stored in the onboard database of processor 111) to an updated setting (which may also be stored in onboard database of processor 111) based upon the desired setting.

Finally, method 401 for controlling a window shade includes step 470, controlling a height of the window shade by utilizing the updated current setting on the onboard database, so as to control the user's sleep cycle. A height of the window shade may also be referred to as a shade height. Step 470 involves processor 111 controlling motor module 120 so that the extendable window shade is adjusted according to the updated current setting. For example, if the updated current setting specifies that the extendable window shade is to be lowered to a shade height of fully-closed at a time of 7:00 PM at half speed, then processor 111 will control motor module 120 to lower the extendable window shade to a shade height of fully-closed at half-speed at 7:00 PM.

While the present invention has been illustrated and described herein in terms of a preferred embodiment and several alternatives, it is to be understood that the systems and methods described herein can have a multitude of additional uses and applications. Accordingly, the invention should not be limited to just the particular description and various drawing figures contained in this specification that merely illustrate a preferred embodiment and application of the principles of the invention.

What is claimed is:

1. A motorized window shade system, comprising:
a shade housing surrounding an extendable window shade, wherein the shade housing includes a processor;
a motor module for controllably adjusting a shade height of the extendable window shade;
a wireless communication device; and
a power supply.

2. The motorized window shade system as recited in claim 1, further comprising a speaker for emitting one or more sounds.

3. The motorized window shade system as recited in claim 1, wherein the processor includes an onboard database for storing a current setting.

4. The motorized window shade system as recited in claim 1, wherein the processor includes an onboard database for storing a current setting, a desired setting, and an updated current setting.

5. The motorized window shade system as recited in claim 1, further comprising a cloud server for communicating with the processor and with a user's mobile device.

6. The motorized window shade system as recited in claim 5, wherein the processor includes an onboard database for storing a current setting, and wherein the cloud server includes a cloud database for storing the current setting.

7. The motorized window shade system as recited in claim 5, wherein the processor includes an onboard database for storing a current setting, a desired setting, and an updated current setting, and wherein the cloud server includes a cloud database for storing the current setting, the desired setting, and the updated current setting.

8. The motorized window shade system as recited in claim 1, wherein the power supply includes a processor power supply and a shade power supply.

9. The motorized window shade system as recited in claim 8, wherein the processor power supply and the shade power supply are physically separate, and wherein the processor power supply supplies power to the processor and the wireless communication device, and the shade power supply supplies power to the motor module.

10. The motorized window shade system as recited in claim 1, wherein the power supply includes a solar panel.

11. The motorized window shade system as recited in claim 10, wherein the solar panel is positioned between the window and the extendable window shade so that the solar panel may receive light regardless of the shade height of the extendable window shade.

12. The motorized window shade system as recited in claim 1, further comprising a photosensor for monitoring a light level of an environment outside the window.

13. The motorized window shade system as recited in claim 3, wherein the processor receives the light level from the photosensor and adjusts the current setting based upon the light level.

14. A method for controlling a window shade to control a user's sleep cycle, comprising the steps of:
connecting to the user's mobile device;
communicating a current setting from an onboard database to the user's mobile device;
receiving a desired setting from the user's mobile device;
updating the current setting from the onboard database to an updated current setting on the onboard database based upon the desired setting; and
controlling a height of the window shade by utilizing the updated current setting on the onboard database, so as to control the user's sleep cycle.

15. The method for controlling a window shade to control a user's sleep cycle as recited in claim 14, further comprising the step of controlling a speaker to emit one or more sounds so as to further control the user's sleep cycle.

16. A method for controlling a window shade to control a user's sleep cycle, comprising the steps of:
connecting to a cloud server;
communicating a current setting from an onboard database to the cloud server;
the cloud server receiving a desired setting from the user's mobile device;
storing the desired setting in the cloud server;
receiving the desired setting from the cloud server;
updating the current setting from the onboard database to an updated current setting on the onboard database based upon the desired setting; and
controlling a height of the window shade by utilizing the updated current setting on the onboard database, so as to control the user's sleep cycle.

17. The method for controlling a window shade to control a user's sleep cycle as recited in claim 16, further comprising the step of controlling a speaker to emit one or more sounds so as to further control the user's sleep cycle.
